(19) United States
(12) Patent Application Publication
(10) Pub. No.: US 2023/0317216 A1
(43) Pub. Date: Oct. 5, 2023

(54) USER INTERFACES FOR ASSISTING IN FORM COMPLETION

(71) Applicant: Lark Technologies, Inc., Mountain View, CA (US)

(72) Inventors: Sam Freund, Washington, DC (US); Corinne Bain, New York City, NY (US)

(21) Appl. No.: 18/120,906

(22) Filed: Mar. 13, 2023

Related U.S. Application Data
(60) Provisional application No. 63/325,677, filed on Mar. 31, 2022.

(51) Int. Cl. G16H 10/20; G16H 50/30

(52) U.S. Cl. CPC G16H 10/20; G16H 50/30

(57) ABSTRACT

Improved user interfaces for assisting in form completion (and/or systems, apparatuses, methods, and/or computer program products that present such improved user interfaces) store data for incomplete forms for people, such as the ASCVD (atherosclerotic cardiovascular disease) risk score questionnaire and/or other questionnaires and/or forms, and remind the people regarding incomplete information while providing assistance in obtaining the incomplete information. In some examples, the reminders and/or assistance may be provided via one or more chatbots. In various examples, the assistance may be tailored for the people using information stored about the people.

### CROSS-REFERENCE TO RELATED APPLICATION

This application is a nonprovisional patent application of and claims the benefit under 35 U.S.C. § 119(e) of U.S. Provisional Patent Application No. 63/325,677, filed Mar. 31, 2022, and titled “User Interfaces for Assisting in Form Completion,” the contents of which are incorporated herein by reference in their entirety.

### FIELD

The described embodiments relate generally to user interfaces. More particularly, the present embodiments relate to improved user interfaces for assisting in form completion.

### BACKGROUND

People complete forms for a variety of reasons. For example, questionnaire forms are often used by a doctor to obtain information from a patient in order to make a medical diagnosis. One such questionnaire is the questionnaire used to determine a patient's ASCVD (atherosclerotic cardiovascular disease) risk score.

### OVERVIEW

The present disclosure relates to providing improved user interfaces for assisting in form completion by storing data for incomplete forms for people, such as the ASCVD (atherosclerotic cardiovascular disease) risk score questionnaire, and reminding the people regarding incomplete information while providing assistance in obtaining the incomplete information. In some examples, the reminders and/or assistance may be provided via one or more chatbots. In various examples, the assistance may be tailored for the people using information stored about the people.

In various embodiments, a system includes a memory allocation and a processor allocation. The memory allocation is configured to store at least one executable asset. The processor allocation is configured to access the memory allocation and execute the at least one executable asset to instantiate a service that provides a form to a person using data from at least one non-transitory storage medium, determines that the person has partially completed the form, records information that the person provided in the at least one non-transitory storage medium, provides a reminder to the person to provide incomplete information, and provides the person assistance to obtain the incomplete information.

In some examples, the form is an ASCVD (atherosclerotic cardiovascular disease) risk score questionnaire. In a number of implementations of such examples, the form requests at least one of the person's age, the person's sex, the person's race, the person's systolic blood pressure, the person's diastolic blood pressure, the person's total cholesterol, the person's HDL (high-density lipoprotein) cholesterol, the person's LDL (low-density lipoprotein) cholesterol, the person's history of diabetes, the person's smoking status, whether or not the person is on hypertension treatment, whether or not the person is on a statin, or whether or not the person is on aspirin therapy. In various implementations of such examples, the service further determines an ASCVD risk score for the person when the form is complete. In some implementations of such examples, the service further categorizes the ASCVD risk score for the person. In a number of implementations of such examples, the service further provides a recommendation according to the ASCVD risk score for the person.

In various examples, the service provides the person the assistance to obtain the incomplete information via at least one chatbot. In some implementations of such examples, the at least one chatbot communicates with the person via an app executing on a mobile computing device associated with the person.

In a number of examples, the service provides the person the assistance to obtain the incomplete information using the information stored for the person. In various examples, the service is operable to receive input from the person indicating that the person possesses the incomplete information. In some implementations of such examples, the service is operable to complete the form in response to receiving the incomplete information from the person after receiving the input.

In various examples, the service provides a reminder to the person to provide the incomplete information according to input that the person provided regarding when to provide the reminder regarding the incomplete information. In some examples, the service retrieves the data from the at least one non-transitory storage medium. In a number of examples, the service determines that the person has partially completed the form by analyzing input the person provided in response to the service providing the form.

In some embodiments, a method includes providing a form to a person using at least one processor and data retrieved from at least one non-transitory storage medium; determining, using the at least one processor, that the person has partially completed the form; recording information that the person provided in the at least one non-transitory storage medium; providing a reminder to the person to provide incomplete information using the at least one processor; and providing, using the at least one processor, the person assistance to obtain the incomplete information.

In a various of examples, the providing the person the assistance includes launching a chatbot. In some examples, the determining that the person has partially completed the form includes determining that the form has at least one blank field.

In a number of embodiments, a computer program product, stored in at least one non-transitory storage medium, includes first instructions, executable by at least one processor to provide a form to a person using data retrieved from the at least one non-transitory storage medium; second instructions, executable by the at least one processor to determine that the person has partially completed the form; third instructions, executable by the at least one processor to record information that the person provided in the at least one non-transitory storage medium; fourth instructions, executable by the at least one processor to provide a reminder to the person to provide incomplete information using the at least one processor; and fifth instructions, executable by the at least one processor to provide the person assistance to obtain the incomplete information.

In some examples, the computer program product further includes sixth instructions, executable by the at least one processor to categorize the person based at least on the information. In various examples, the providing the form includes transmitting the data to a mobile electronic device associated with the person.

### BRIEF DESCRIPTION OF THE DRAWINGS

The disclosure will be readily understood by the following detailed description in conjunction with the accompanying drawings, wherein like reference numerals designate like structural elements.

FIG. 1 depicts a first example system for improved user interfaces that assist in form completion.

FIG. 2 depicts a flow chart illustrating a first example method for providing improved user interfaces that assist in form completion. This method may be performed by the system of FIG. 1.

### DETAILED DESCRIPTION

Reference will now be made in detail to representative embodiments illustrated in the accompanying drawings. It should be understood that the following descriptions are not intended to limit the embodiments to one preferred embodiment. To the contrary, it is intended to cover alternatives, modifications, and equivalents as can be included within the spirit and scope of the described embodiments as defined by the appended claims.

The description that follows includes sample systems, methods, apparatuses, and computer program products that embody various elements of the present disclosure. However, it should be understood that the described disclosure may be practiced in a variety of forms in addition to those described herein.

The ASCVD (atherosclerotic cardiovascular disease) risk score questionnaire asks information regarding a patient's age, sex, race, systolic blood pressure, diastolic blood pressure, total cholesterol, HDL (high-density lipoprotein) cholesterol, LDL (low-density lipoprotein) cholesterol, history of diabetes, smoking status, whether or not the patient is on hypertension treatment, whether or not the patient is on a statin, and whether or not the patient is on aspirin therapy. This information is used to determine a patient's 10 year ASCVD risk. Some (such as blood pressure and cholesterol) and/or all of this information may be required for generating the ASCVD risk score such that an incomplete questionnaire may prevent score calculation. An ASCVD risk score of <5% may be categorized as a low risk. An ASCVD risk score of 5%-7.5% may be categorized as a borderline risk. An ASCVD risk score of 7.5-20% may be categorized as an intermediate risk. An ASCVD risk score of ≥20% may be categorized as a high risk. High risk individuals may be strongly recommended statin therapy on the basis of risk alone after a clinician patient risk discussion. Conversely, those in the low risk category may not benefit from statin therapy and may focus on healthy habits for CVD (cardiovascular disease) prevention. The remaining groups, which may be classified as borderline or intermediate risk, may be recommended statin therapy and/or healthy habits based on the ASCVD risk score and/or other information.

Questionnaires, such as the ASCVD risk score questionnaire, are typically administered in an in-person medical visit by medical personnel because they often involve information that very few patients would have at the ready (such as blood pressure readings, cholesterol, and so on). In an in-person medical visit scenario, medical personnel are present to answer patient questions and assist the patient in obtaining the necessary information. However, in-person medical visits are expensive and time consuming.

Questionnaires may be automated as forms so that patients may complete the questionnaires via an app, web site, and/or similar interface that communicates with one or more servers and/or other backends. This may greatly expand the number of patients who may complete such questionnaires due to not requiring an in-person medical visit. However, such a distributed technical solution adds its own technical problem in that medical personnel are not present to answer patient questions, assist the patient in obtaining the necessary information, and/or assist the patient in understanding the flow of data collection for completing such questionnaires. In some situations, patients may not be able to complete forms due to missing information since medical personnel are not present to answer patient questions and assist the patient in obtaining the missing information. As a result, the technical problem introduced by the distributed technical solution may prevent form completion and lose some or all of the advantages in patient reach that the distributed technical solution introduced.

The present disclosure may solve this problem by storing data for incomplete forms for people, such as the ASCVD risk score questionnaire and/or other questionnaires, and reminding the people regarding incomplete information while providing assistance in obtaining the incomplete information. In some examples, the reminders and/or assistance may be provided via one or more chatbots. In various examples, the assistance may be tailored for the people using information stored about the people.

Unlike conventional solutions, in some implementations, the present disclosure may allow people to submit partially completed data collection forms and subsequently guide them through alternative means to collect each of the rest of the required data items individually as a follow-up. This may be provided in the course of artificial intelligence and/or other automated health support, which may be configured to help people with one or more programs (overarching courses of treatment or actions for a person, designed to help with one or more conditions) (such as one or more heart health programs). Rather than through error messages that a form is incomplete, the people may be prompted in the form of a more natural conversation and may be reminded to do so several times during the course of a normal program interaction, resulting in an improved user interface for the people. After the people signal that they have successfully collected the needed data elements, they may be brought back to the form, which they may then complete (such as to receive their ASCVD risk score). As such, the present disclosure provides an unconventional technical solution to the technical problem introduced by the distributed technical solution to questionnaire completion discussed above.

In this way, the present disclosure may provide improved user interfaces for assisting in form completion. Further, systems that implement the present disclosure may be enabled to perform functions that the systems would not previously have been able to perform absent the technology disclosed herein. This may enable the system to operate more efficiently while consuming fewer hardware and/or software resources as more resource consuming techniques, such as those involving actual medical personnel interaction with the people, could be omitted. This may enable reduction of unnecessary hardware and/or software components and provide greater system flexibility. Further, by recording partially completed form information and assisting in later completion, the present disclosure may prevent people from partially completing and then later completely re-completing the forms. This may result in usage of fewer hardware and/or software resources as people may not repeatedly partially complete forms that are then never used.

The present disclosure may provide improved user interfaces for assisting in form completion by storing data for incomplete forms for people, such as the ASCVD risk score questionnaire, and reminding the people regarding incomplete information while providing assistance in obtaining the incomplete information. In some examples, the reminders and/or assistance may be provided via one or more chatbots. In various examples, the assistance may be tailored for the people using information stored about the people.

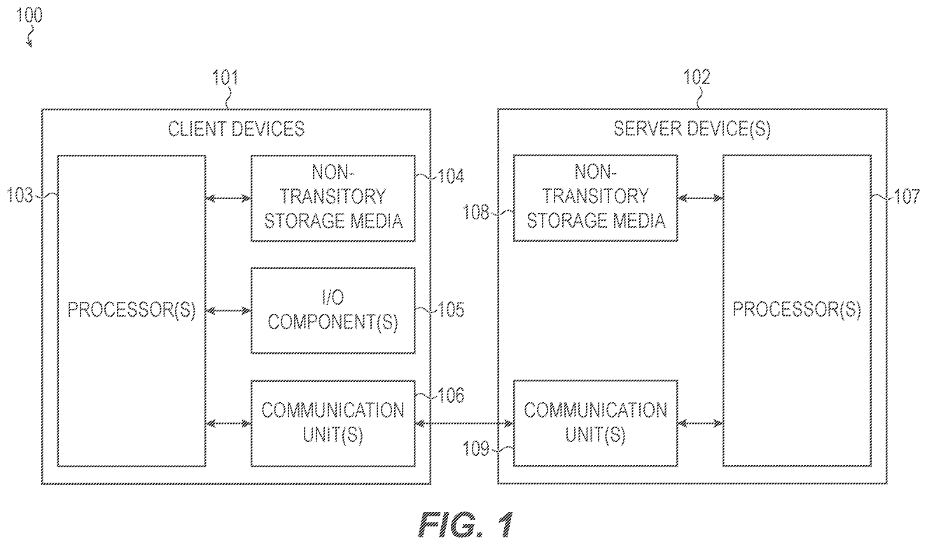

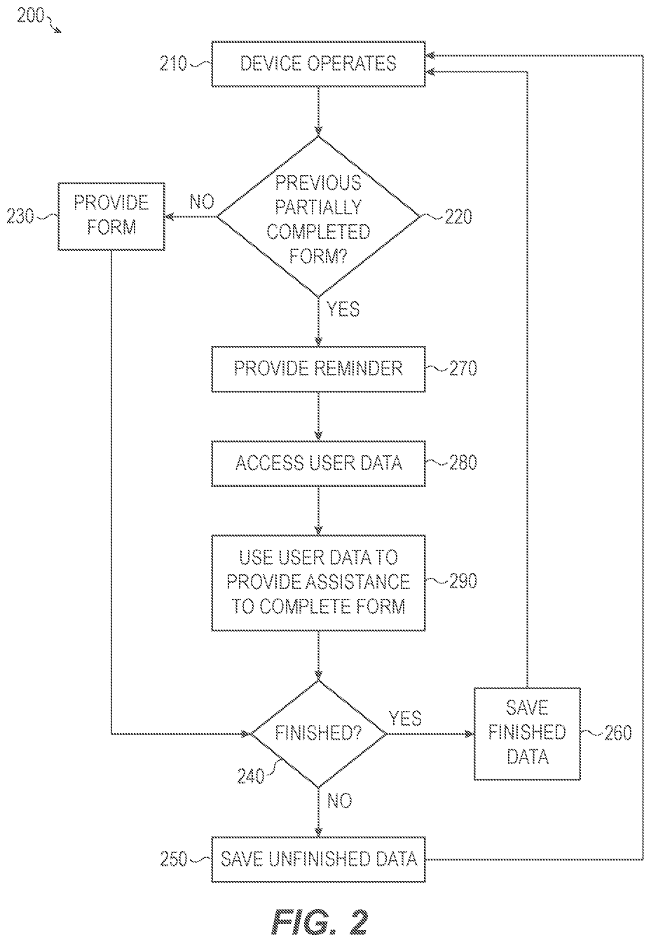

These and other embodiments are discussed below with reference to FIGS. 1-2. However, those skilled in the art will readily appreciate that the detailed description given herein with respect to these Figures is for explanatory purposes only and should not be construed as limiting.

FIG. 1 depicts a first example system 100 for improved user interfaces that assist in form completion. The system 100 may provide improved user interfaces for assisting in form completion by storing data for incomplete forms for people, such as the ASCVD risk score questionnaire, and reminding the people regarding incomplete information while providing assistance in obtaining the incomplete information. In some examples, the system 100 may provide the reminders and/or assistance via one or more chatbots. In various examples, the system 100 may tailor the assistance for the people using information stored about the people. The system 100 may include one or more client devices 101 and one more server devices 102 that may be operable to communicate with each other via one or more wired and/or wireless communication networks.

The client device 101 may be operable to provide output to one or more people (such as one or more forms that may be formatted for display using data retrieved from the non-transitory storage medium 104, received from the server device 102, and so on), obtain input from the one or more people, store data, communicate with the server device 102, and/or perform various processing. The server device 102 may provide code to the client device 101 to execute, provide data to the client device 101 (such as data for one or more forms retrieved from the non-transitory storage medium 108), store data, perform various backend processing, and so on. Processing that may be performed by either and/or both of the client device 101 and the server device 102 may include storing data for incomplete forms for people, reminding the people regarding incomplete information while providing assistance in obtaining the incomplete information, implementing one or more chatbots, tailoring the assistance for the people using information stored about the people, providing artificial intelligence and/or other automated health support (such as one or more heart health programs), and so on.

For example, as part of providing artificial intelligence and/or other automated health support and/or separate from such, the system 100 may provide an ASCVD risk score questionnaire (and/or another questionnaire and/or form) to a person. As part of such, the system may ask the person for information regarding the patient's age, sex, race, systolic blood pressure, diastolic blood pressure, total cholesterol, HDL (high-density lipoprotein) cholesterol, LDL (low-density lipoprotein) cholesterol, history of diabetes, smoking status, whether or not the person is on hypertension treatment, whether or not the person is on a statin, and whether or not the person is on aspirin therapy. Upon completion, the system 100 may use this information to determine the person's 10 year ASCVD risk.

The system 100 may categorize an ASCVD risk score of <5% as a low risk. The system 100 may categorize an ASCVD risk score of 5%-7.5% as a borderline risk. The system 100 may categorize an ASCVD risk score of 7.5-20% as an intermediate risk. The system 100 may categorize an ASCVD risk score of ≥20% as a high risk. The system 100 may make one or more specific, targeted treatment recommendations based upon the categorization. The system 100 may recommend high risk individuals talk to a medical professional about statin therapy. Conversely, the system 100 may recommend people in the low risk category to focus on healthy habits for CVD prevention (such as a heart healthy diet, smoking cessation, maintaining a healthy weight, appropriate exercise, and so on). The system 100 may recommend that people in the remaining groups, which may be classified as borderline or intermediate risk, statin therapy and/or healthy habits based on their ASCVD risk score and/or other information.

However, the person may indicate to the system 100 that the person does not possess all of the information and/or the system 100 may otherwise determine that the person has partially competed the ASCVD risk score questionnaire and/or other questionnaire and/or form (such as by analyzing the input provided by the person in response to the ASCVD risk score questionnaire and/or other questionnaire and/or form and determining that it is incomplete, that not all questions are answered, that there are one or more blank fields, and so on). The system 100 may then record the information that has been provided and provide a reminder to the person to provide such. In some implementations, the system 100 may obtain input from the person as to when to provide a reminder to the person about the incomplete information. The system 100 may also provide assistance in obtaining the incomplete information. Various configurations are possible and contemplated without departing from the scope of the present disclosure.

For example, the incomplete information may be systolic blood pressure and/or diastolic blood pressure. The system 100 may provide assistance in obtaining the incomplete information by providing information regarding what systolic blood pressure and/or diastolic blood pressure are, why systolic blood pressure and/or diastolic blood pressure measurements are needed, how systolic blood pressure and/or diastolic blood pressure are tested, where systolic blood pressure and/or diastolic blood pressure tests can be obtained, and so on. Various configurations are possible and contemplated without departing from the scope of the present disclosure.

In some examples, the system 100 may provide the reminders and/or assistance via one or more chatbots. Various configurations are possible and contemplated without departing from the scope of the present disclosure. Here is a sample conversation of how a chatbot may provide reminders and/or assistance:

Chatbot: Hi! You previously partially completed a ASCVD risk score form, but did not finish the blood pressure portion. Would you like assistance?

Person: Yes.

Chatbot: Blood pressure is the pressure of circulating blood against the walls of blood vessels. Most of this pressure results from the heart pumping blood through the circulatory system. Blood pressure is one of the key vital signs that medical professionals use in evaluating a patient's health. A medical professional can measure blood pressure quickly and non-invasively by listening through a stethoscope for sounds in a patient's arm's artery as the artery is squeezed. Automated machines for quickly and non-invasively measuring blood pressure are often found at neighborhood pharmacies. Would you like a list of pharmacies?

Person: Yes.

Chatbot: Please provide your zip code for a list of pharmacies within twenty miles that have automated blood pressure machines.

In various examples, system 100 may tailor the assistance for the people using information stored about the people. For example, the system 100 may store information regarding a person's health plan provider and/or insurer, a person's address, and/or other information. Upon determining that the person has incomplete information for a form, such as the person's cholesterol, the system 100 may direct the person to a site for the person's health plan provider or insurer where the person may find the person's cholesterol if the person has had their cholesterol recently tested via the person's health plan provider or insurer. Alternatively and/or additionally, the system 100 may use the stored information on the person's health plan provider or insurer to determine where the person can get a covered cholesterol test and/or assist the person with scheduling such a test. Further, alternatively and/or additionally, the system 100 may use the stored address for the person to provide labs close to the person that offer cholesterol testing. Various configurations are possible and contemplated without departing from the scope of the present disclosure.

The client device 101 may be any kind of electronic device. Examples of such devices include, but are not limited to, one or more desktop computing devices, laptop computing devices, server computing devices, mobile computing devices, tablet computing devices, set top boxes, digital video recorders, televisions, displays, wearable devices, smart phones, digital media players, and so on. The client device 101 may include one or more processors 103 and/or other processing units and/or controllers, one or more non-transitory storage media 104 (which may take the form of, but is not limited to, a magnetic storage medium; optical storage medium; magneto-optical storage medium; read only memory; random access memory; erasable programmable memory; flash memory; and so on), one or more input and/or output components 105 (such as one or more displays, touch screens, keyboards, computer mice, haptic devices, microphones, speakers, printers, and so on), one or more communication units 106, and/or other components. The processor 103 may execute instructions stored in the non-transitory storage medium 104 to perform various functions. Such functions may include providing output to one or more people, obtaining input from the one or more people, storing data, communicating with the server device 102, performing various processing, and so on. Alternatively and/or additionally, the client device 101 may involve one or more memory allocations configured to store at least one executable asset and one or more processor allocations configured to access the one or more memory allocations and execute the at least one executable asset to instantiate one or more processes and/or services, such as one or more artificial intelligence and/or other automated health support services, one or more form services, one or more form completion services, and so on.

Similarly, the server device 102 may be any kind of electronic device. Examples of such devices include, but are not limited to, one or more desktop computing devices, laptop computing devices, server computing devices, mobile computing devices, tablet computing devices, set top boxes, digital video recorders, televisions, displays, wearable devices, smart phones, digital media players, and so on. The server device 102 may include one or more processors 107 and/or other processing units and/or controllers, one or more non-transitory storage media 108 (which may take the form of, but is not limited to, a magnetic storage medium; optical storage medium; magneto-optical storage medium; read only memory; random access memory; erasable programmable memory; flash memory; and so on), one or more communication units 109, and/or other components. The processor 107 may execute instructions stored in the non-transitory storage medium 108 to perform various functions. Such functions may include providing code to the client device 101 to execute, storing data, performing various backend processing, and so on. Alternatively and/or additionally, the server device 102 may involve one or more memory allocations configured to store at least one executable asset and one or more processor allocations configured to access the one or more memory allocations and execute the at least one executable asset to instantiate one or more processes and/or services, such as one or more artificial intelligence and/or other automated health support services, one or more form services, one or more form completion services, and so on.

As used herein, the term “computing resource” (along with other similar terms and phrases, including, but not limited to, “computing device” and “computing network”) refers to any physical and/or virtual electronic device or machine component, or set or group of interconnected and/or communicably coupled physical and/or virtual electronic devices or machine components, suitable to execute or cause to be executed one or more arithmetic or logical operations on digital data.

Example computing resources contemplated herein include, but are not limited to: single or multi-core processors; single or multi-thread processors; purpose-configured co-processors (e.g., graphics processing units, motion processing units, sensor processing units, and the like); volatile or non-volatile memory; application-specific integrated circuits; field-programmable gate arrays; input/output devices and systems and components thereof (e.g., keyboards, mice, trackpads, generic human interface devices, video cameras, microphones, speakers, and the like); networking appliances and systems and components thereof (e.g., routers, switches, firewalls, packet shapers, content filters, network interface controllers or cards, access points, modems, and the like); embedded devices and systems and components thereof (e.g., system(s)-on-chip, Internet-of-Things devices, and the like); industrial control or automation devices and systems and components thereof (e.g., programmable logic controllers, programmable relays, supervisory control and data acquisition controllers, discrete controllers, and the like); vehicle or aeronautical control devices and systems and components thereof (e.g., navigation devices, safety devices or controllers, security devices, and the like); corporate or business infrastructure devices or appliances (e.g., private branch exchange devices, voice-over internet protocol hosts and controllers, end-user terminals, and the like); personal electronic devices and systems and components thereof (e.g., cellular phones, tablet computers, desktop computers, laptop computers, wearable devices); personal electronic devices and accessories thereof (e.g., peripheral input devices, wearable devices, implantable devices, medical devices and so on); and so on. It may be appreciated that the foregoing examples are not exhaustive.

Example information can include, but may not be limited to: personal identification information (e.g., names, social security numbers, telephone numbers, email addresses, physical addresses, driver's license information, passport numbers, and so on); identity documents (e.g., driver's licenses, passports, government identification cards or credentials, and so on); protected health information (e.g., medical records, dental records, and so on); financial, banking, credit, or debt information; third-party service account information (e.g., usernames, passwords, social media handles, and so on); encrypted or unencrypted files; database files; network connection logs; shell history; filesystem files; libraries, frameworks, and binaries; registry entries; settings files; executing processes; hardware vendors, versions, and/or information associated with the compromised computing resource; installed applications or services; password hashes; idle time, uptime, and/or last login time; document files; product renderings; presentation files; image files; customer information; configuration files; passwords; and so on. It may be appreciated that the foregoing examples are not exhaustive.

The foregoing examples and description of instances of purpose-configured software, whether accessible via API as a request-response service, an event-driven service, or whether configured as a self-contained data processing service are understood as not exhaustive. In other words, a person of skill in the art may appreciate that the various functions and operations of a system such as described herein can be implemented in a number of suitable ways, developed leveraging any number of suitable libraries, frameworks, first or third-party APIs, local or remote databases (whether relational, NoSQL, or other architectures, or a combination thereof), programming languages, software design techniques (e.g., procedural, asynchronous, event-driven, and so on or any combination thereof), and so on. The various functions described herein can be implemented in the same manner (as one example, leveraging a common language and/or design), or in different ways. In many embodiments, functions of a system described herein are implemented as discrete microservices, which may be containerized or executed/instantiated leveraging a discrete virtual machine, that are only responsive to authenticated API requests from other microservices of the same system. Similarly, each microservice may be configured to provide data output and receive data input across an encrypted data channel. In some cases, each microservice may be configured to store its own data in a dedicated encrypted database; in others, microservices can store encrypted data in a common database; whether such data is stored in tables shared by multiple microservices or whether microservices may leverage independent and separate tables/schemas can vary from embodiment to embodiment. As a result of these described and other equivalent architectures, it may be appreciated that a system such as described herein can be implemented in a number of suitable ways. For simplicity of description, many embodiments that follow are described in reference to an implementation in which discrete functions of the system are implemented as discrete microservices. It is appreciated that this is merely one possible implementation.

As described herein, the term “processor” refers to any software and/or hardware-implemented data processing device or circuit physically and/or structurally configured to instantiate one or more classes or objects that are purpose-configured to perform specific transformations of data including operations represented as code and/or instructions included in a program that can be stored within, and accessed from, a memory. This term is meant to encompass a single processor or processing unit, multiple processors, multiple processing units, analog or digital circuits, or other suitably configured computing element or combination of elements.

Although the system 100 is illustrated and described as including particular components arranged in a particular configuration, it is understood that this is an example. In a number of implementations, various configurations of various components may be used without departing from the scope of the present disclosure.

For example, the system 100 is illustrated and described as including both the client device 101 and the server device 102. However, it is understood that this is an example. In various implementations, the system 100 may include a client device 101 that is operable to perform one or more of the above functions without involvement of the server device 102. Various configurations are possible and contemplated without departing from the scope of the present disclosure.

FIG. 2 depicts a flow chart illustrating a first example method 200 for providing improved user interfaces that assist in form completion. This method 200 may be performed by the system 100 of FIG. 1.

At operation 210, an electronic device may operate. The electronic device may be the client device 101 of FIG. 1, the server device 102 of FIG. 1, and/or another device.

At operation 220, the electronic device may determine whether or not it stores and/or has access to data for a previously partially completed form. The form may correspond to an ASCVD risk score questionnaire and the data may correspond to one or more of a person's age, the person's sex, the person's race, the person's systolic blood pressure, the person's diastolic blood pressure, the person's total cholesterol, the person's HDL cholesterol, the person's LDL cholesterol, the person's history of diabetes, the person's smoking status, whether or not the person is on hypertension treatment, whether or not the person is on a statin, and whether or not the patient is on aspirin therapy. If so, the flow may proceed to operation 270. Otherwise, the flow may proceed to operation 230.

At operation 230, after the electronic device determines that it does not store and/or have access to data for a previously partially completed form, the electronic device may provide the form. The flow may then proceed to operation 240 where the electronic device may receive input for the form and determine whether or not there is any incomplete information for the form. As discussed above, the form may correspond to an ASCVD risk score questionnaire and the incomplete information may correspond to one or more of a person's age, the person's sex, the person's race, the person's systolic blood pressure, the person's diastolic blood pressure, the person's total cholesterol, the person's HDL cholesterol, the person's LDL cholesterol, the person's history of diabetes, the person's smoking status, whether or not the person is on hypertension treatment, whether or not the person is on a statin, and whether or not the patient is on aspirin therapy. If not, the flow may proceed to operation 260 and the electronic device may record the finished data (as well as generate any scores based on the form and/or provide any recommendations based thereon) before the flow returns to operation 210 and the electronic device continues to operate. Otherwise, the flow may proceed to operation 250 and the electronic device may record the unfinished data (as well as any other task, such as obtaining any user input regarding setting one or more reminders for completing the form) before the flow returns to operation 210 and the electronic device continues to operate.

At operation 270, after the electronic device determines that it does store and/or have access to data for a previously partially completed form, the electronic device may provide one or more reminders regarding completing the form. As discussed above, the form may correspond to an ASCVD risk score questionnaire and the incomplete information may correspond to one or more of a person's age, the person's sex, the person's race, the person's systolic blood pressure, the person's diastolic blood pressure, the person's total cholesterol, the person's HDL cholesterol, the person's LDL cholesterol, the person's history of diabetes, the person's smoking status, whether or not the person is on hypertension treatment, whether or not the person is on a statin, and whether or not the patient is on aspirin therapy. Such reminders may be based upon input that the person provided.

The flow may then proceed to operation 280 where the electronic device may access user data. The user data may be information regarding a person's health plan provider and/or insurer, a person's address, and/or other information. The flow may then proceed to operation 290 where the electronic device may provide assistance to complete the form.

For example, the electronic device may store information regarding a person's health plan provider and/or insurer, a person's address, and/or other information. Upon determining that the person has incomplete information for a form, such as the person's cholesterol, the electronic device may direct the person to a site for the person's health plan provider or insurer where the person may find the person's cholesterol if the person has had their cholesterol recently tested via the person's health plan provider or insurer. Alternatively and/or additionally, the electronic device may use the stored information on the person's health plan provider or insurer to determine where the person can get a covered cholesterol test and/or assist the person with scheduling such a test. Further, alternatively and/or additionally, the electronic device may use the stored address for the person to provide labs close to the person that offer cholesterol testing. Various configurations are possible and contemplated without departing from the scope of the present disclosure.

Subsequently, the flow may then proceed to operation 240 where the electronic device may receive input for the form to determine whether or not there is any incomplete information for the form.

In various examples, this example method 200 may be implemented as a group of interrelated software modules or components that perform various functions discussed herein. These software modules or components may be executed within a cloud network and/or by one or more computing devices, such as the client device 101 and/or the server device 102 of FIG. 1.

Although the example method 200 is illustrated and described as including particular operations performed in a particular order, it is understood that this is an example. In various implementations, various orders of the same, similar, and/or different operations may be performed without departing from the scope of the present disclosure.

For example, the method 200 is illustrated and described as the electronic device receiving the incomplete data from the person after the electronic device provides the assistance. However, it is understood that this is an example. In various implementations, the electronic device may be operable to receive the incomplete information from the person anytime the person indicates that the person has come into possession of the incomplete information. Various configurations are possible and contemplated without departing from the scope of the present disclosure.

By way of another example, the method 200 is illustrated and described as accessing user data that may be used to provide assistance to complete the form. However, it is understood that this is an example. In various implementations, the electronic device may not access such user data and/or may not use such user data to provide assistance to complete the form. Various configurations are possible and contemplated without departing from the scope of the present disclosure.

Although the above illustrates and describes a number of embodiments, it is understood that these are examples. In various implementations, various techniques of individual embodiments may be combined without departing from the scope of the present disclosure.

In various implementations, a system may include a memory allocation and a processor allocation. The memory allocation may be configured to store at least one executable asset. The processor allocation may be configured to access the memory allocation and execute the at least one executable asset to instantiate a service that provides a form to a person using data from at least one non-transitory storage medium, determines that the person has partially completed the form, records information that the person provided in the at least one non-transitory storage medium; provides a reminder to the person to provide incomplete information, and provides the person assistance to obtain the incomplete information.

In some examples, the form may be an ASCVD (atherosclerotic cardiovascular disease) risk score questionnaire. In a number of such examples, the form may request at least one of the person's age, the person's sex, the person's race, the person's systolic blood pressure, the person's diastolic blood pressure, the person's total cholesterol, the person's HDL (high-density lipoprotein) cholesterol, the person's LDL (low-density lipoprotein) cholesterol, the person's history of diabetes, the person's smoking status, whether or not the person is on hypertension treatment, whether or not the person is on a statin, or whether or not the patient is on aspirin therapy. In various such examples, the service further may determine an ASCVD risk score for the person when the form is complete. In some such examples, the service may further categorize the ASCVD risk score for the person. In a number of such examples, the service may further provide a recommendation according to the ASCVD risk score for the person.

In various examples, the service may provide the person the assistance to obtain the incomplete information via at least one chatbot. In some such examples, the at least one chatbot may communicate with the person via an app executing on a mobile computing device associated with the person.

In a number of examples, the service may provide the person the assistance to obtain the incomplete information using the information stored for the person. In various examples, the service may be operable to receive input from the person indicating that the person possesses the incomplete information. In some such examples, the service may be operable to complete the form in response to receiving the incomplete information from the person after receiving the input.

In various examples, the service may provide a reminder to the person to provide the incomplete information according to input that the person provided regarding when to provide the reminder regarding the incomplete information. In some examples, the service may retrieve the data from the at least one non-transitory storage medium. In a number of examples, the service may determine that the person has partially completed the form by analyzing input the person provided in response to the service providing the form.

In some implementations, a method may include providing a form to a person using at least one processor and data retrieved from at least one non-transitory storage medium; determining, using the at least one processor, that the person has partially completed the form; recording information that the person provided in the at least one non-transitory storage medium; providing a reminder to the person to provide incomplete information using the at least one processor; and providing, using the at least one processor, the person assistance to obtain the incomplete information.

In various examples, the providing the person the assistance may include launching a chatbot. In some examples, the determining that the person has partially completed the form may include determining that the form has at least one blank field.

In a number of implementations, a computer program product, stored in at least one non-transitory storage medium, may include first instructions, executable by at least one processor to provide a form to a person using data retrieved from the at least one non-transitory storage medium; second instructions, executable by the at least one processor to determine that the person has partially completed the form; third instructions, executable by the at least one processor to record information that the person provided in the at least one non-transitory storage medium; fourth instructions, executable by the at least one processor to provide a reminder to the person to provide incomplete information using the at least one processor; and fifth instructions, executable by the at least one processor to provide the person assistance to obtain the incomplete information.

In some examples, the computer program product may further include sixth instructions, executable by the at least one processor to categorize the person based at least on the information. In various examples, the providing the form may include transmitting the data to a mobile electronic device associated with the person.

As described above and illustrated in the accompanying figures, the present disclosure relates to providing improved user interfaces for assisting in form completion by storing data for incomplete forms for people, such as the ASCVD risk score questionnaire, and reminding the people regarding incomplete information while providing assistance in obtaining the incomplete information. In some examples, the reminders and/or assistance may be provided via one or more chatbots. In various examples, the assistance may be tailored for the people using information stored about the people.

In the present disclosure, the methods disclosed may be implemented as sets of instructions or software readable by a device. Further, it is understood that the specific order or hierarchy of steps in the methods disclosed are examples of sample approaches. In other embodiments, the specific order or hierarchy of steps in the method can be rearranged while remaining within the disclosed subject matter. The accompanying method claims present elements of the various steps in a sample order, and are not necessarily meant to be limited to the specific order or hierarchy presented.

The described disclosure may be provided as a computer program product, or software, that may include a non-transitory machine-readable medium having stored thereon instructions, which may be used to program a computer system (or other electronic devices) to perform a process according to the present disclosure. A non-transitory machine-readable medium includes any mechanism for storing information in a form (e.g., software, processing application) readable by a machine (e.g., a computer). The non-transitory machine-readable medium may take the form of, but is not limited to, a magnetic storage medium (e.g., floppy diskette, video cassette, and so on); optical storage medium (e.g., CD-ROM); magneto-optical storage medium; read only memory (ROM); random access memory (RAM); erasable programmable memory (e.g., EPROM and EEPROM); flash memory; and so on.

The foregoing description, for purposes of explanation, used specific nomenclature to provide a thorough understanding of the described embodiments. However, it will be apparent to one skilled in the art that the specific details are not required in order to practice the described embodiments. Thus, the foregoing descriptions of the specific embodiments described herein are presented for purposes of illustration and description. They are not targeted to be exhaustive or to limit the embodiments to the precise forms disclosed. It will be apparent to one of ordinary skill in the art that many modifications and variations are possible in view of the above teachings.

What is claimed is:

1. A system, comprising:
a memory allocation configured to store at least one executable asset; and
a processor allocation configured to access the memory allocation and execute the at least one executable asset to instantiate:
a service that:
provides a form to a person using data from at least one non-transitory storage medium;
determines that the person has partially completed the form;
records information that the person provided in the at least one non-transitory storage medium;
provide a reminder to the person to provide incomplete information; and
provides the person assistance to obtain the incomplete information.

2. The system of claim 1, wherein the form is an ASCVD (atherosclerotic cardiovascular disease) risk score questionnaire.

3. The system of claim 2, wherein the form requests at least one of:
the person's age;
the person's sex;
the person's race;
the person's systolic blood pressure;
the person's diastolic blood pressure;
the person's total cholesterol;
the person's HDL (high-density lipoprotein) cholesterol;
the person's LDL (low-density lipoprotein) cholesterol;
the person's history of diabetes;
the person's smoking status;
whether or not the person is on hypertension treatment;
whether or not the person is on a statin; or
whether or not the patient is on aspirin therapy.

4. The system of claim 2, wherein the service further determines an ASCVD risk score for the person when the form is complete.

5. The system of claim 4, wherein the service further categorizes the ASCVD risk score for the person.

6. The system of claim 5, wherein the service further provides a recommendation according to the ASCVD risk score for the person.

7. The system of claim 1, wherein the service provides the person the assistance to obtain the incomplete information via at least one chatbot.

8. The system of claim 7, wherein the at least one chatbot communicates with the person via an app executing on a mobile computing device associated with the person.

9. The system of claim 1, wherein the service provides the person the assistance to obtain the incomplete information using the information stored for the person.

10. The system of claim 1, wherein the service is operable to receive input from the person indicating that the person possesses the incomplete information.

11. The system of claim 10, wherein the service is operable to complete the form in response to receiving the incomplete information from the person after receiving the input.

12. The system of claim 1, wherein the service provides the reminder to the person to provide the incomplete information according to input that the person provided regarding when to provide the reminder regarding the incomplete information.

13. The system of claim 1, wherein the service retrieves the data from the at least one non-transitory storage medium.

14. The system of claim 1, wherein the service determines that the person has partially completed the form by analyzing input the person provided in response to the service providing the form.

15. A method, comprising:
providing a form to a person using at least one processor and data retrieved from at least one non-transitory storage medium;
determining, using the at least one processor, that the person has partially completed the form;
recording information that the person provided in the at least one non-transitory storage medium;
providing a reminder to the person to provide incomplete information using the at least one processor; and
providing, using the at least one processor, the person assistance to obtain the incomplete information.

16. The method of claim 15, wherein the providing the person the assistance comprises launching a chatbot.

17. The method of claim 15, wherein the determining that the person has partially completed the form comprises determining that the form has at least one blank field.

18. A computer program product, stored in at least one non-transitory storage medium, comprising:
first instructions, executable by at least one processor to provide a form to a person using data retrieved from the at least one non-transitory storage medium;
second instructions, executable by the at least one processor to determine that the person has partially completed the form;
third instructions, executable by the at least one processor to record information that the person provided in the at least one non-transitory storage medium;
fourth instructions, executable by the at least one processor to provide a reminder to the person to provide incomplete information using the at least one processor; and
fifth instructions, executable by the at least one processor to provide the person assistance to obtain the incomplete information.

19. The computer program product of claim 18, further comprising sixth instructions, executable by the at least one processor to categorize the person based at least on the information.

20. The computer program product of claim 18, wherein the providing the form comprises transmitting the data to a mobile electronic device associated with the person.
